(19) United States
(12) Patent Application Publication
(10) Pub. No.: US 2023/0364106 A1
(43) Pub. Date: Nov. 16, 2023

(54) PARENTERAL DOSAGE FORM OF DILTIAZEM

(71) Applicant: SUN PHARMACEUTICAL INDUSTRIES LIMITED, Mumbai, Maharashtra (IN)

(72) Inventors: Samarth KUMAR, Baroda (IN); Milan Mohanbhai VASOYA, Baroda (IN); Devendra Pratap SINGH, Baroda (IN); Ajay Jaysingh KHOPADE, Baroda (IN); Subhas Balaram BHOWMICK, Baroda (IN)

(21) Appl. No.: 18/027,252

(22) PCT Filed: Sep. 21, 2021
(86) PCT No.: PCT/IB2021/058587

§ 371 Date: Mar. 20, 2023

(30) Foreign Application Priority Data
Sep. 21, 2020 (IN) 202021040712

(51) Int. Cl. A61K 31/554; A61K 47/26; A61K 9/00

(52) U.S. Cl. CPC A61K 31/554; A61K 47/26; A61K 9/0029

(57) ABSTRACT

A parenteral dosage form of diltiazem, comprising a ready-to-infuse, stable aqueous solution comprising diltiazem or its pharmaceutically acceptable salt, a pharmaceutically acceptable stabilizer selected from cyclic oligosaccharides, and an infusion container fdled with the said aqueous solution.

### CROSS REFERENCE

The present patent application claims the benefit of the priority date of Indian Provisional Patent Application No. 202021040712 filed on Sep. 21, 2020.

### FIELD OF THE INVENTION

The present invention relates to a parenteral dosage form of diltiazem comprising a ready-to-infuse, stable aqueous solution of diltiazem which can be administered to a patient in need thereof without manipulations in terms of its concentration and which is stable for a prolonged period of time.

### BACKGROUND OF THE INVENTION

Diltiazem is a calcium ion influx inhibitor (slow channel blocker or calcium channel antagonist). Chemically, diltiazem hydrochloride is 1,5-benzothiazepin-4(5H)one,3-(acetyloxy)-5-[2-(dimethylamino)ethyl]-2,3-dihydro-2-(4-methoxyphenyl)-, monohydro-chloride, (+)-cis- and has the following structural formula:

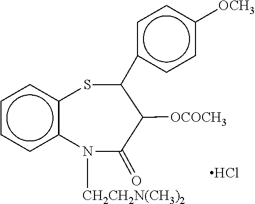

Diltiazem, inhibits the influx of calcium (Ca) ions during membrane depolarization of cardiac and vascular smooth muscle. Diltiazem hydrochloride is administered by intravenous infusion for temporary control of rapid ventricular rate in atrial fibrillation or atrial flutter and rapid conversion of paroxysmal supraventricular tachycardias (PSVT) to sinus rhythm.

The commercially available injectable products of diltiazem are pre-concentrate solutions which need to be diluted with a suitable diluent like dextrose or sodium chloride solution before use. One such product by Bedford® is a diltiazem hydrochloride 5 mg/ml injection solution, supplied in 5 ml and 10 ml vials. The step of dilution and handling involves risk of potential calculation or dilution error as well as risk of microbiological contamination during handling. Further, diltiazem is known to be susceptible to hydrolysis and degradation in aqueous solutions. The degradation is undesirable as it results in loss of titer of the active ingredient, and leads to formation of impurities or related compounds which have negligible activity and are undesirable. The major pathway of degradation is o-deacetylation which leads to formation of impurity “desacetyl diltiazem HCl”.

Thus, there is a need in the art for a stable parenteral dosage form of diltiazem, which comprise an aqueous solution of diltiazem that is ready-to-infuse and can be administered without any manipulation, i.e., in the pre-diluted form that can be directly infused or injected thus eliminating the risk of any potential calculation or dilution error as well as risk of microbiological contamination during handling and at the same time is stable for a prolonged period of time. The present invention fulfills this need.

It was a surprise finding of the present inventors that presence of cyclic oligosaccharide in parenteral dosage forms of diltiazem provides a significant stability. The inventors have found that an aqueous solution of diltiazem is more stable in the presence of cyclic oligosaccharides, which tend to stabilize diltiazem in aqueous solution and protect it from degradation.

### SUMMARY OF THE INVENTION

The present invention relates to a parenteral dosage form comprising a ready-to-infuse, stable aqueous solution comprising diltiazem or its pharmaceutically acceptable salt and a cyclic oligosaccharide as a stabilizer.

In one aspect, the present relates to a parenteral dosage form comprising a ready-to-infuse, stable aqueous solution comprising diltiazem or its pharmaceutically acceptable salt, a cyclic oligosaccharide stabilizer, a pH-adjusting agent, and an alcoholic solvent.

In yet another aspect, the present invention provides a parenteral dosage form comprising a ready-to-infuse, stable aqueous solution comprising diltiazem or its pharmaceutically acceptable salt and a cyclic oligosaccharide stabilizer and in said parenteral dosage form the level of desacetyl diltiazem or its salt impurity is not more than 10% w/w of diltiazem or its pharmaceutically acceptable salt when stored at 2-8° C. for at least 12 months or at 25° C140% RH for at least 6 months.

In another aspects, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein in the dosage form the level of desacetyl diltiazem or its salt impurity is not more than 6% w/w of diltiazem or its pharmaceutically acceptable salt when stored at 2-8° C. for at least 12 months or at 25° C./40% RH for at least 6 months.

### DESCRIPTION OF THE INVENTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

The present invention provides a parenteral dosage form comprising a ready-to-infuse, stable aqueous solution comprising diltiazem or its pharmaceutically acceptable salt and a cyclic oligosaccharide as a stabilizer.

The present invention relates to an aqueous solution of diltiazem that is ready-to-infuse without any manipulation, i.e., in the pre-diluted form that can be directly infused or injected thus eliminating the risk of any potential calculation or dilution error as well as risk of microbiological contamination during handling and at the same time is stable for a prolonged period of time. More particularly, the invention relates to a stable, ready-to-infuse, aqueous parenteral dosage form comprising diltiazem or its pharmaceutically acceptable salt, a pH-adjusting agent to provide a pH in the range of 3.5 to 5.0, at least one pharmaceutically acceptable stabilizer selected from a cyclic oligosaccharide and an infusion container filled with said aqueous solution.

The term “diltiazem” as used herein includes diltiazem as well as its pharmaceutically acceptable salts, such as diltiazem hydrochloride and other pharmaceutically acceptable salts or derivative thereof.

The term “ready-to-infuse” as used herein means that the aqueous drug solution is sterile and suitable for direct intravenous infusion or injection without manipulation, that is, no intermediate steps of dilution, reconstitution, dispensing, sterilization, transfer, handling or compounding are required before administration or infusion of the drug solution to the patient. The aqueous drug solution can be directly administered parenterally from the container of the dosage form. The term “ready-to-infuse” is synonymous with “ready-to-inject” or “ready-to-administer” or “directly administering” or “direct intravenous infusion” or “direct delivery” or ready to use. The ready-to-infuse parenteral dosage form according to the present invention avoids the inconvenience of reconstituting or diluting a lyophilized or concentrated parenteral formulation into infusion diluents prior to infusion, as well as the risk of any potential calculation or dilution error as well as risk of microbiological contamination during handling. Also, a ready-to-infuse parenteral dosage form can be said to be a premixed dosage form which can be administered directly without any dilution or mixing requirement. Moreover, diltiazem is an emergency life-saving medicine and hence reduced time in getting the dose already in ready-to-infuse dosage forms is advantageous and preferred. The present invention provides a stable parenteral dosage form of diltiazem having a ready-to-infuse aqueous solution of diltiazem that does not relate to semi-solid topical dosage forms (such as gel, hydrogel, emulgel, paste, cream, ointment, etc.) and/or non-aqueous dosage forms that are not suitable for parenteral administration.

The parenteral dosage form of the present invention is “stable”. As used herein, the term “stable” means that the dosage form of the present invention is physically as well as chemically stable upon storage at refrigerated conditions (2-8° C.), for prolonged period of time, such as for at least 6 months, preferably 12 months, more preferably for 18 months to 24 months. The solution is also stable when stored at 25° C. (room temperature condition) for a period of at least 3 months, preferably 6 months, more preferably for 9 months to 12 months. When stored at these conditions, the aqueous solution of diltiazem or its pharmaceutically acceptable salt remains chemically stable, wherein various parameters such a drug content (assay of diltiazem) and content of related substances, i.e., known impurities, unknown impurities and total impurity remains within specified limits. Suitably, the assay of diltiazem remains within 90%-110% by weight of the label claim, the content of total impurities (excluding desacetyl diltiazem) remain within 1% w/w of diltiazem hydrochloride and the content of impurity ‘desacetyl diltiazem HCl’ remains within 10% by weight of diltiazem or its pharmaceutically acceptable salt, preferably within 6% by weight of diltiazem hydrochloride. The impurities are expressed as % by weight of diltiazem or its pharmaceutically acceptable salt.

In some embodiments, the parenteral dosage form of the present invention comprises diltiazem HCl salt and the assay of diltiazem remains within 90%-110% by weight of the label claim, and the content of total impurities (excluding desacetyl diltiazem HCl) remain within 1% w/w of diltiazem hydrochloride and the content of impurity ‘desacetyl diltiazem HCl’ remains within 10% by weight of diltiazem hydrochloride, preferably within 6% by weight of diltiazem hydrochloride. The impurities are expressed as % by weight of diltiazem hydrochloride.

The term “cyclic oligosaccharide” as used here in includes compounds with macrocyclic ring of glucose subunits joined by α-1,4 glycosidic bonds such as cyclodextrins. Cyclodextrins are classified as natural and derived cyclodextrins. Natural cyclodextrins include three well-known industrially produced (major and minor) cyclic oligosaccharides. The most common natural cyclodextrins are α, β, and γ consisting of 6, 7, and 8 glucopyranose units. Hydroxypropyl-β-cyclodextrin (HP-β-CD), randomly methylated-β-cyclodextrin (RM-β-CD), and sulfobutylether-β-cyclodextrin (SBE-β-CD) are mostly preferred for complexation.

The parenteral dosage form of the present invention is sterile. The term “sterile” or “sterilized” as used herein, means that the aqueous solution has been brought to a state of sterility and has not been subsequently exposed to microbiological contamination, i.e., the sterility of the solution present in the container has not been compromised. The solution complies with the sterility requirements of the standard Pharmacopoeias, such as the United States Pharmacopoeias (USP). Sterilization may be achieved by suitable techniques such as filtration sterilization, radiation sterilization, steam sterilization and the like. In one embodiment, the disclosed parenteral dosage form is subjected to sterilization by autoclaving the dosage form at ≥121° C. for 15 minutes.

In one embodiment, the parenteral dosage form according to the present invention comprises of a ready-to-infuse, stable aqueous solution comprising diltiazem or its pharmaceutically acceptable salt and a cyclic oligosaccharide as a stabilizer, wherein the cyclic oligosaccharide comprises a macrocyclic ring of glucose subunits joined by α-1,4 glycosidic bonds.

In another embodiment, the parenteral dosage form according to the present invention comprises of a ready-to-infuse, stable aqueous solution comprising diltiazem or its pharmaceutically acceptable salt and a cyclic oligosaccharide as a stabilizer, wherein said dosage form is filled in an infusion container.

In yet another embodiment, the parenteral dosage form according to the present invention comprises of a ready-to-infuse, stable aqueous solution comprising diltiazem or its pharmaceutically acceptable salt and a cyclic oligosaccharide as a stabilizer filled in an infusion container, wherein said infusion container is selected from an infusion bag, perfusion bag, flexible pouch, soft bag, infusion bottle or pre-filled syringe.

In some embodiment, the disclosed stable parenteral dosage form according to the present invention further comprises an organic solvent. In some embodiment, the organic solvent is an alcoholic solvent.

In one embodiment, the parenteral dosage form comprises a ready-to-infuse, stable aqueous solution comprising diltiazem or its pharmaceutically acceptable salt and a cyclic oligosaccharide as a stabilizer and an alcoholic solvent.

In some embodiments, the disclosed stable parenteral dosage form comprises at least one pharmaceutically acceptable stabilizer that is a cyclic oligosaccharide. The cyclic oligosaccharide includes, but is not limited to, hydroxypropyl derivatives of cyclodextrin (such as hydroxypropyl-(3-cyclodextrin (HP-β-CD)), randomly methylated-β-cyclodextrin (RM-β-CD), and sulfobutylether-β-cyclodextrin (SBE-β-CD). In some embodiments, the hydroxypropyl derivative of cyclodextrin is hydroxypropyl-β-cyclodextrin (HP-β-CD). In some embodiment of the present invention, the cyclic oligosaccharide used in the present invention is hydroxypropyl-β-cyclodextrin, methylated-β-cyclodextrin (RM-β-CD) or sulfobutylether-β-cyclodextrin (SBE-β-CD). Preferably, the cyclic oligosaccharide used in the present invention is hydroxypropyl-β-cyclodextrin (HP-β-CD).

In an embodiment of the present invention, the parenteral dosage form comprises a ready-to-infuse, stable aqueous solution comprising diltiazem or its pharmaceutically acceptable salt, a cyclic oligosaccharide as a stabilizer, a pH-adjusting agent, and an alcoholic solvent.

In another embodiment, the parenteral dosage form comprises a ready-to-infuse, stable aqueous solution comprising diltiazem or its pharmaceutically acceptable salt, a cyclic oligosaccharide as a stabilizer and a pH-adjusting agent to provide a pH in the range of about 3.5 to about 5.

In one embodiment, the parenteral dosage form comprises a ready-to-infuse, stable aqueous solution comprising diltiazem or its pharmaceutically acceptable salt, a cyclic oligosaccharide as a stabilizer, a pH-adjusting agent, and an alcoholic solvent, wherein the cyclic oligosaccharide is selected from hydroxypropyl-β-cyclodextrin (HP-β-CD), methylated-β-cyclodextrin (RM-β-CD) or sulfobutylether-β-cyclodextrin (SBE-β-CD).

In some embodiments according to the present invention, the cyclic oligosaccharide in the parenteral dosage form is present at a concentration ranging from about 0.1% to about 100% w/v. In one embodiment, the cyclic oligosaccharide is present at a concentration ranging from about 0.1% to about 50% w/v. In a further embodiment, the cyclic oligosaccharide is present at a concentration of about 5% to about 40% w/v. In yet another embodiment, the cyclic oligosaccharide is present at a concentration of about 8% to about 35% w/v.

In an embodiment, the parenteral dosage form comprises a ready-to-infuse, stable aqueous solution comprising diltiazem or its pharmaceutically acceptable salt, a cyclic oligosaccharide as a stabilizer, a pH-adjusting agent, and an alcoholic solvent, wherein the alcoholic solvent is ethanol or a mixture of ethanol with another co-solvent.

In yet another embodiment, the present invention provides a stable, ready-to-infuse, aqueous parenteral dosage form comprising diltiazem or its pharmaceutically acceptable salt, a pH-adjusting agent to provide a pH in the range of about 3.5 to about 5, and at least one pharmaceutically acceptable stabilizer selected from hydroxypropyl derivatives of cyclodextrin.

In a further embodiment, the present invention provides a stable, ready-to-infuse, aqueous parenteral dosage form comprising diltiazem or its pharmaceutically acceptable salt, a pH-adjusting agent to provide a pH in the range of about 3.5 to about 5, at least one pharmaceutically acceptable stabilizer selected from hydroxypropyl derivatives of cyclodextrin and an infusion container filled with said aqueous solution.

In yet another embodiment, the present invention provides a stable, ready-to-infuse, aqueous parenteral dosage form comprising diltiazem or its pharmaceutically acceptable salt, a pH-adjusting agent to provide a pH in the range of about 3.7 to about 4.5, at least one pharmaceutically acceptable hydroxypropyl-β-cyclodextrin stabilizer and an infusion container filled with said aqueous solution.

In one embodiment, the present invention provides a stable, ready-to-infuse, aqueous parenteral dosage form comprising diltiazem or its pharmaceutically acceptable salt, a pH-adjusting agent to provide a pH in the range of about 3.7 to about 4.5, at least one pharmaceutically acceptable hydroxypropyl-β-cyclodextrin stabilizer and at least one alcoholic solvent.

In another embodiment, the present invention provides a stable, ready-to-infuse, aqueous parenteral dosage form comprising diltiazem or its pharmaceutically acceptable salt, a pH-adjusting agent to provide a pH in the range of about 3.7 to about 4.5, at least one pharmaceutically acceptable hydroxypropyl-β-cyclodextrin stabilizer, at least one alcoholic solvent, and an infusion container filled with said aqueous solution.

In yet another embodiment, the present invention provides a stable, ready-to-infuse, aqueous parenteral dosage form comprising diltiazem or its pharmaceutically acceptable salt, a pH-adjusting agent to provide a pH in the range of about 3.7 to about 4.5, at least one pharmaceutically acceptable hydroxypropyl-β-cyclodextrin stabilizer, ethanol and an infusion container filled with said aqueous solution.

In one embodiment, hydroxypropyl-β-cyclodextrin (“HP-β-CD”) is present at a concentration ranging from about 0.1% to about 50% w/v. In a further embodiment, HP-β-CD is present at a concentration ranging from about 1% to about 50% w/v. In a yet further embodiment, the concentration of HP-β-CD is from about 1% to about 10% w/v.

In some embodiment, hydroxypropyl-β-cyclodextrin is present at a concentration of about 3.5% w/v. In another embodiment, hydroxypropyl-β-cyclodextrin is present at a concentration of about 1% w/v.

In one embodiment of the present invention, the infusion container of the dosage form is filled with an aqueous solution comprising diltiazem or its pharmaceutically acceptable salt as the active ingredient. Diltiazem or its pharmaceutically acceptable salt is present in the aqueous solution of the present invention at a concentration which allows direct infusion of the aqueous solution to the patient without the need of further dilution. It may be present at a concentration ranging from about 0.05 mg/ml to about 2.0 mg/ml, preferably from about 0.1 mg/ml to about 2.0 mg/ml, such as for example 0.2, 0.3, 0.4, 0.5, 0.6, 0.7, 0.8, 0.9, 1.0, 1.1, 1.2, 1.3, 1.4, 1.5, 1.6, 1.7, 1.8 or 1.9 mg/ml, more preferably from about 0.4 mg/ml to about 1.0 mg/ml. In one particular embodiment, diltiazem hydrochloride is present in the aqueous solution in an amount of about 1.0 mg/ml.

In some embodiments, the parenteral dosage form of the present invention includes the aqueous solution of diltiazem filled into infusion container which may be rigid or flexible in nature. The volume capacity of each unit of the container may range from about 50 ml to about 500 ml. The aqueous solution may present in the infusion containers in volumes ranging from about 50 ml to about 500 ml per infusion container, such as for example 50 ml, 75 ml, 100 ml, 120 ml, 125 ml, 140 ml, 150 ml, 160 ml, 175 ml, 180 ml, 190 ml, 200 ml, 220 ml, 225 ml, 240 ml, 250 ml, 260 ml, 275 ml, 280 ml, 290 ml, 300 ml, 320 ml, 325 ml, 340 ml, 350 ml, 360 ml, 375 ml, 380 ml, 390 ml, 400 ml, 420 ml, 425 ml, 430 ml, 440 ml, 450 ml, 460 ml, 470 ml, 475 ml, 480 ml, 490 ml or 500m1. According to preferred embodiments of the present invention, the ready-to-infuse parenteral dosage form provides large volume containers such as infusion bags, which can accommodate a volume of at least 50 ml, preferably from about 100 ml to about 500 ml of the aqueous solution.

In another embodiment, the present invention provides a parenteral dosage form comprising a ready-to-infuse, stable aqueous solution comprises diltiazem or its pharmaceutical acceptable salt in a concentration range of about 0.05 to 2.0 mg/ml, a cyclic oligosaccharide stabilizer in a concentration range of about 0.1 to 50% w/v, and the pH of the solution is in range of pH 3.7 to 4.5.

In another embodiment, the present invention provides a parenteral dosage form comprising a ready-to-infuse, stable aqueous solution comprises diltiazem or its pharmaceutical acceptable salt in a concentration range of about 0.1 to 2.0 mg/ml, a cyclic oligosaccharide stabilizer, such as hydroxypropyl-β-cyclodextrin, in a concentration range of about 0.1 to 10% w/v, and the pH of the solution is pH 3.9.

In yet another embodiment, the present invention provides a parenteral dosage form comprising a ready-to-infuse, stable aqueous solution comprises diltiazem or its pharmaceutical salt in a concentration range of about 0.1 to 2.0 mg/ml, a cyclic oligosaccharide, such as hydroxypropyl-β-cyclodextrin, in a concentration range of about 0.1 to 10% w/v, ethanol in a concentration range of about 1 to 10% w/w and the pH of the solution is pH 3.9.

In one embodiment, the stable parenteral dosage form of the present invention further comprises an organic solvent, preferably an alcoholic solvent. In related embodiments, the alcoholic solvent used in present invention is ethanol and it may be present in the aqueous solution of the present invention in an amount ranging from about 1.0% w/v to about 50% w/v, preferably from about 1.0% w/v to about 20.0% w/v, more preferably from about 1.0% w/v to about 15.0% w/v, such as for example 1.0, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14,14.5 or 15% w/v. In one particularly preferred embodiment, the ethanol is present in the ready-to-infuse aqueous solution in an amount ranging from about 1.0% w/v to about 5.0% w/v, preferably about 1.35% w/v. In one preferred embodiment, the aqueous solution of the present invention comprises ethanol as a co-solvent. In another embodiment, the aqueous solution of the present invention comprises a mixture of ethanol and other alcohols or solvents as a co-solvent.

In a further embodiment, the parenteral dosage form or the ready-to-infuse aqueous solution of the present invention further comprises other parentally acceptable excipients. Parentally acceptable excipients that may be used include, but are not limited to, pH-adjusting agents and buffers, osmogen or osmotic/tonicity adjusting agents, chelating agents, etc. In one preferred embodiment, the dosage form is free of anti-oxidants and preservatives.

In another embodiment of the present invention, various other components which may be used in the composition according to the invention includes butylated hydroxyanisole, butylated hydroxytoluene, ammonium sulphate, sodium metabisulfite, edetate disodium, hydroxypropyl betadex, L-methionine, potassium phosphate monobasic, anhydrous lactose, betadex sulfobutyl ether sodium, crospovidone, dextran 40, glycerin, pentetic acid, poloxamer 188, polyethylene glycol 300, polyethylene glycol 3350, polyethylene glycol 400, polyethylene glycol 4000, polyethylene glycol 600, povidone, povidone K12, povidone K15, propylene glycol, tartaric acid (granular), L-cysteine hydrochloride monohydrate, phosphoric acid, ammonium sulphate, propyl paraben, boric acid, sodium metabisulfite granular, ascorbic acid, benzoic acid, benzyl alcohol, L-arginine, lactic acid, methyl paraben, polyoxyl 35 castor oil (Cremophor® ELP), polysorbate-80, polysorbate-20, potassium phosphate monobasic crystal, sodium dihydrogen phosphate monohydrate, soybean oil, aspartic acid, glutamic acid and HCl. These components may individually or combined promote the overall stability of the formulation for long-term storage.

The pH of the aqueous solution may be adjusted in the desired range by use of a pH-adjusting agent. The pH-adjusting agent includes, but is not limited to, buffering agents known in the art. The pH-adjusting and/or buffering agents that may be used in the present invention include, but are not limited to, citric acid, sodium citrate, sodium hydroxide, hydrochloric acid, sulfuric acid, acetic acid, sodium acetate, tartaric acid, potassium hydroxide and the like and mixtures thereof. In one embodiment, the pH may be auto-adjusted in the desired range by the ingredients present in the solution of the present invention. The pH of the solution ranges from about 3.5 to about 5, in some aspects the pH of the solution is about 3.7 to about 4.5, such as for example 3.7, 3.8, 3.9, 4.0, 4.1, 4.2, 4.3 or 4.4, and more preferably from about 3.7 to about 4.3.

In an embodiment, the dosage form according to present invention may be stable with or without a buffer component, and may also be used as such without any additional pH adjustment.

In one preferred embodiment, the ready-to-infuse solution of diltiazem comprises or consists essentially of a citric acid or citrate buffer to adjust and maintain the pH in the range of about 3.7 to about 4.5. The ready-to-infuse aqueous solution of the present invention is iso-osmolar to the parenteral/plasma fluids. The tonicity adjusting agent or osmogen that may be used may be selected from, but are not limited to, mannitol, dextrose, sucrose, sorbitol, glycerin, glycerol, sucrose, xylitol, fructose, mannose, maltitol, inositol, trehalose, sodium chloride, potassium chloride, calcium chloride, magnesium chloride, inorganic salts, urea and the like and mixtures thereof. In one embodiment, the osmogen/tonicity adjusting agent according to the present invention comprises dextrose, sodium chloride, sorbitol, mannitol or ethanol.

In some embodiments according to the present invention, the tonicity adjusting agent in the parenteral dosage form is present at a concentration ranging from about 0.01% to about 50% w/v. In one embodiment, tonicity adjusting agent is present at a concentration ranging from about 0.1% to about 35% w/v. In a further embodiment, tonicity adjusting agent is present at a concentration of about 1% to about 20% w/v. In yet another embodiment, cyclic oligosaccharide is present at a concentration of about 1% to about 10% w/v.

In one embodiment, the present invention provides a parenteral dosage form comprising a ready-to-infuse, stable aqueous solution comprises Diltiazem or its pharmaceutical acceptable salt in a concentration range of about 0.05 to 2.0 mg/ml, a cyclic oligosaccharide stabilizer in a concentration range of about 0.1 to 50% w/v, and the pH of the solution is in range of pH 3.7 to 4.5, wherein when said aqueous solution is stored at 2-8° C. for at least 12 months, the level of desacetyl diltiazem or its salt impurity is less than 10% by weight of diltiazem or its pharmaceutically acceptable salt.

In another embodiment, the ready-to-infuse parenteral dosage form according to present invention comprises diltiazem or its pharmaceutically acceptable salt and a cyclic oligosaccharide as a stabilizer, wherein the dosage form level of desacetyl diltiazem or its salt impurity is not more than 10% w/w of diltiazem or its pharmaceutically acceptable salt when stored for at least 12 months at 2-8° C.

In another embodiment, the ready-to-infuse parenteral dosage form according to present invention comprises diltiazem or its pharmaceutically acceptable salt and a cyclic oligosaccharide as a stabilizer, wherein in the dosage form the level of desacetyl diltiazem or its salt impurity is not more than 10% w/w of diltiazem or its pharmaceutically acceptable salt when stored for at least 6 months at 25° C./40% RH.

In some embodiments, the present invention provides a parenteral dosage form comprising a ready-to-infuse, stable aqueous solution of diltiazem or its pharmaceutical acceptable salt, wherein in the dosage form the level of desacetyl diltiazem or its salt impurity is less than 10% w/w, preferably less than 8% w/w, more preferably less than 6% w/w of diltiazem or its pharmaceutically acceptable salt when stored at 2-8° C. for at least 12 months.

In some embodiments, the present invention provides a parenteral dosage form comprising a ready-to-infuse, stable aqueous solution of diltiazem or its pharmaceutical acceptable salt, wherein in the dosage form the level of desacetyl diltiazem or its salt impurity is less than 10% w/w, preferably less than 8% w/w, more preferably less than 6% w/w of diltiazem or its pharmaceutically acceptable salt when stored at 25° C./40% RH for at least 6 months.

In one embodiment, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein in the dosage form the level of desacetyl diltiazem or its salt impurity is not more than 6% w/w of diltiazem or its pharmaceutically acceptable salt when stored for at least 12 months at 2-8° C.

In one embodiment, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein in the dosage form the level of desacetyl diltiazem or its salt impurity is not more than 6% w/w of diltiazem or its pharmaceutically acceptable salt when stored for at least 6 months at 25° C./40% RH.

In one embodiment, the present invention provides a parenteral dosage form comprising a ready-to-infuse, stable aqueous solution comprises diltiazem or its pharmaceutical acceptable salt in a concentration range of about 0.05 to 2.0 mg/ml, a cyclic oligosaccharide stabilizer in a concentration range of about 0.1 to 50% w/v, and the pH of the solution is in range of pH 3.7 to 4.5, wherein when said aqueous solution is stored at 25° C./40% RH for at least 6 months, the level of desacetyl diltiazem or its salt impurity is less than 6% by weight of diltiazem or its pharmaceutically acceptable salt.

In another embodiment according to present invention, the total impurity level (excluding desacetyl diltiazem HCl) in the ready-to-infuse parenteral dosage form is not more than 1% and the desacetyl diltiazem impurity is not more than 6% w/w of diltiazem or its pharmaceutically acceptable salt when stored at 2-8° C. for at least 12 months.

In another embodiment according to present invention, the total impurity level (excluding desacetyl diltiazem HCl) in the ready-to-infuse parenteral dosage is not more than 1% and desacetyl diltiazem impurity is not more than 6% w/w of diltiazem or its pharmaceutically acceptable salt when stored at 25° C./40% RH for at least 6 months.

In one embodiment, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein the cyclic oligosaccharide comprises a macrocyclic ring of glucose subunits joined by α-1,4 glycosidic bonds.

In one embodiment, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein the said dosage form is filled in an infusion container.

In some embodiments, the cyclic oligosaccharide is selected from hydroxypropyl-β-cyclodextrin, methylated-β-cyclodextrin (RM-β-CD) or sulfobutylether-β-cyclodextrin (SBE-β-CD). In a further embodiment, the cyclic oligosaccharide is hydroxypropyl-β-cyclodextrin (HP-β-CD). In one embodiment, the cyclic oligosaccharide stabilizer is a cyclodextrin.

In some embodiments, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer and an alcoholic solvent or a mixture thereof. In one embodiment, the organic solvent is an alcoholic solvent. In one embodiment, the alcoholic solvent is ethanol.

In one embodiment, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer and an alcoholic solvent or a mixture thereof, wherein the method further comprises a pH adjustment step using a suitable pH-adjusting agent to provide a pH in the range of 3.5-5.

In some embodiments, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein in the dosage form the level of desacetyl diltiazem or its salt impurity is not more than 6% w/w of diltiazem or its pharmaceutically acceptable salt when stored for at least 12 months at 2-8° C. and wherein said method further comprises terminally sterilizing the dosage form by autoclaving.

In some embodiments, the present invention provides a method for controlling the level of desacetyl diltiazem or its salt impurity in a ready-to-infuse parenteral dosage form of diltiazem or its pharmaceutically acceptable salt by using a cyclic oligosaccharide stabilizer, wherein in the dosage form the level of desacetyl diltiazem or its salt impurity is not more than 6% w/w of diltiazem or its pharmaceutically acceptable salt when stored for at least 6 months at 25° C./40% RH and wherein said method further comprises terminally sterilizing the dosage form by autoclaving.

In one embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 12 months at 2-8° C.

In another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 6 months at 25° C./40% RH.

In a further embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w, preferably less than 8% w/w, more preferably less than 6% w/w of diltiazem or its pharmaceutically acceptable salt when stored at 2-8° C. for at least 12 month.

In one further embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w, preferably less than 8% w/w, more preferably less than 6% w/w of diltiazem or its pharmaceutically acceptable salt when stored at 25° C./40% RH for at least 6 months.

In yet another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 12 months at 2-8° C., wherein the cyclic oligosaccharide comprises a macrocyclic ring of glucose subunits joined by α-1,4 glycosidic bonds.

In another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 6 months at 25° C./40% RH, wherein the cyclic oligosaccharide comprises a macrocyclic ring of glucose subunits joined by α-1,4 glycosidic bonds.

In some embodiments, the cyclic oligosaccharide is selected from hydroxypropyl-β-cyclodextrin, methylated-β-cyclodextrin (RM-β-CD) or sulfobutylether-β-cyclodextrin (SBE-β-CD).

In one embodiment, the present invention provides the use of a cyclic oligosaccharide stabilizer for the preparation of a ready-to-infuse, stable aqueous solution of diltiazem or its pharmaceutical acceptable salt, wherein said solution is filled in an infusion container.

In some embodiments, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 12 months at 2-8° C. and the cyclic oligosaccharide stabilizer is a cyclodextrin.

In some embodiments, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 6 months at 25° C./40% RH and the cyclic oligosaccharide stabilizer is a cyclodextrin.

In another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w, preferably less than 8% w/w, more preferably less than 6% w/w of diltiazem or its pharmaceutically acceptable salt when stored at 2-8° C. for at least 12 months.

In another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w, preferably less than 8% w/w, more preferably less than 6% w/w of diltiazem or its pharmaceutically acceptable salt when stored at 25° C./40% RH for at least 6 months.

In some embodiments, the present invention provides the use of a cyclic oligosaccharide stabilizer for the preparation of a ready-to-infuse, stable aqueous solution of diltiazem or its pharmaceutical acceptable salt, wherein the method further comprises addition of an alcoholic solvent or a mixture thereof. In one embodiment, the alcoholic solvent is ethanol.

In another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 12 months at 2-8° C., and said method further comprises the addition of an alcoholic solvent or a mixture thereof, and a pH adjustment step using a suitable pH-adjusting agent to provide a pH in the range of about 3.5 to about 5

In one further embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 6 months at 25° C./40% RH, and said use further comprises the addition of an alcoholic solvent or a mixture thereof, and a pH adjustment step using a suitable pH-adjusting agent to provide a pH in the range of about 3.5 to about 5.

In one embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 12 months at 2-8° C., and said method further comprises terminally sterilizing the dosage form by autoclaving.

In another embodiment, the present invention provides the use of a cyclic oligosaccharide stabilizer for the preparation of a ready-to-infuse, stable aqueous solution of diltiazem or its pharmaceutical acceptable salt, wherein in the solution the level of desacetyl diltiazem or its salt impurity is less than 10% w/w of diltiazem or its pharmaceutically acceptable salt when stored for at least 6 months at 25° C./40% RH, and said method further comprises terminally sterilizing the dosage form by autoclaving.

In one embodiment the present invention provides a parenteral dosage form comprising a ready-to-infuse, stable aqueous solution comprised of diltiazem or its pharmaceutically acceptable salt, wherein the level of desacetyl diltiazem or its salt impurity is not more than 6% w/w of diltiazem or its pharmaceutically acceptable salt when stored for at least 6 months at 25° C./40% RH.

In one embodiment, the present invention provides a parenteral dosage form comprising a ready-to-infuse, stable aqueous solution comprised of diltiazem or its pharmaceutically acceptable salt, wherein the level of desacetyl diltiazem or its salt impurity is not more than 6% w/w of diltiazem or its pharmaceutically acceptable salt when stored for at least 12 months at 2-8° C.

In some embodiments, the stable, ready-to-infuse, aqueous parenteral dosage form of the present invention is terminally sterilized.

In one embodiment, the stable, ready-to-infuse, aqueous parenteral dosage form of the present invention may be terminally sterilized at temperature more than or equal to temperature 121° C. for more than or equal to 15 minutes, or at temperature more than or equal to 121° C. for more than 12 minutes, or at temperature more than 115° C. for more than 8 minutes, or at temperature more than 110° C. for more than 8 minutes, or at temperature not more than 110° C. for more than 8 minutes.

In some embodiments, the stable, ready-to-infuse, aqueous parenteral dosage form of the present invention is terminally sterilized at 121° C. for 15 minutes.

In yet another embodiment, the stable, ready-to-infuse, aqueous parenteral dosage form of the present invention may not be terminally sterilized.

In some embodiments, the infusion container of the parenteral dosage form of the present invention is a flexible infusion container, made up of a flexible material such as plastic or any other polymeric material. In one or more embodiments, the flexible infusion container may be an infusion bag or flexible pouch or soft bag or infusion bottle or film and the like. In another embodiment, the infusion container is a pre-filled syringe. The container may include one or more layers of such materials. Suitably, such materials may include but are not limited to, polyolefin polymers, polyethylene, polypropylene; cyclo olefin polymers, cyclo olefin copolymers, polypropylene based polyolefin polymers; polycarbonates; modified polyolefin-polyethylene polymers or styrene-polyolefin based polymers or block co-polymers thereof. Particularly, the flexible infusion container is not impermeable in nature and possesses some permeation characteristics and the aqueous solution of diltiazem remains in contact with these materials of the container throughout the shelf life of the dosage form.

In some embodiments, the disclosed parenteral dosage form includes the aqueous solution of diltiazem filled into an infusion container, which may be rigid or flexible in nature.

In one embodiment, the flexible infusion containers may be made up of a material comprising a polymer of cyclic olefin such as cyclooolefin homopolymer or cycloolefin copolymer or mixture thereof. Specifically, in a particular embodiment, the container comprises an inner layer made up of a cycloolefin polymer, a middle layer made up of linear low density polyethylene polymer and an outer layer made up of low density polyethylene polymer. Such containers are available commercially. and have a water vapour transmission rate of 2 g /(m²/day) when measured at (40° C./90% relative humidity); oxygen transmission rate of 570 ml/(m².24hour.atm) when measured at (23° C./0% relative humidity) and carbon dioxide transmission rate of 3400 ml/(m².24hour.atm) when measured at 23° C./0% relative humidity.

In another embodiment, the flexible infusion containers may be made up of an outer layer of polypropylene polymer with styrene-ethylene-butylene (SEB) block copolymer and a middle and inner layer made up of polypropylene based polyolefin polymer with styrene-ethylene butylene block copolymer. Such containers are available commercially and have a water vapour transmission rate of 0.62 g/(m²/day) when measured at 23° C./60% relative humidity; oxygen permeability of 1110 ml/(m².24hour.atm) when measured at 23° C./40% relative humidity and carbon dioxide transmission rate of 5149 ml/(m².24hour.atm). Alternatively, the flexible container is made up of multilayer polyolefin film having layers from outside to inside made up of CPET-Tie-PE-Tie-EPC. These containers generally have a water vapour transmission rate of 5.0 g/(m²/day) when measured at 38° C./100% relative humidity; oxygen transmission rate of 1315 cm³/(m².24hour.atm) when measured at 73° F./0% relative humidity and carbon dioxide transmission rate of 3945 cm³/(m².24hour.atm).

In another embodiment, the infusion containers may include an infusion port, which may act as an infusion connector having three assembled parts including a central stopper made up of chlorobutyl or bromobutyl rubber (latex free); an upper breakable part and a bottom part, both made up of polycarbonate. In one embodiment, the infusion container contains a delivery port end for insertion of an infusion set cannula/needle. In an embodiment, the infusion container/bag and the delivery port connecting to the infusion needle form a system whereby during administration of the solution to the patient the vacuum created by outgress of solution is accommodated by the elasticity or flexibility of the infusion bag instead of ingress of external non-sterile air. The dosage form can advantageously maintain the sterility of the solution until it reaches the patient.

In one embodiment, the flexible infusion container includes a thermally resealable portion that is fusible in response to thermal energy, and a container body having a sealed empty chamber in fluid communication with the resealable portion for receiving therein the aqueous solution of the present invention. The method of filling the container includes penetrating the resealable portion with an injection member and introducing the aqueous solution of the present invention into the chamber, withdrawing the injection member while engaging the base of the body to substantially prevent axial movement of the body, and applying thermal energy to the resealable portion to thermally fuse the penetrated region thereof. Such systems are elaborated in U.S. Pat. No. 7,992,597, which is incorporated herein by reference. Further the flexible infusion container may include a chamber for receiving aqueous solution of the present invention and a thermoplastic portion in fluid communication with the chamber. The thermoplastic portion defines a penetrable region that is penetrable by a filling member and is heat resealable to hermetically seal an aperture therein by applying laser radiation at a predetermined wavelength and power and in a predetermined time period. Such systems are elaborated in U.S. Pat. No. 7,490,639, which is incorporated herein by reference.

In yet another embodiment, the flexible infusion container includes a sealed chamber; a first penetrable septum in fluid communication with the chamber that is formed of an elastic material and is penetrable by a first injection member to fill the first chamber with the aqueous solution of the present invention there through; and a second penetrable septum movable between first and second positions. In the first position, at least a portion of the second septum is spaced away from the first septum to allow the injection member to penetrate the first septum and aseptically or sterile fill the chamber with the aqueous solution of the present invention there through. In the second position, the portion of the second septum overlies and seals a resulting injection aperture in the first septum after withdrawal of the first injection member therefrom, and is penetrable by a second injection member to penetrate the first and second septums and withdraw the filled aqueous solution of the present invention from the chamber and through the second injection member. Such systems are elaborated in United States Patent Publication No.US20130333796, which is incorporated herein by reference.

In one embodiment, the infusion container is rigid and is made up of a material such as glass. Such infusion containers include infusion vials, infusion bottles, or pre-filled syringes. However, in preferred embodiments, the container does not have a material that contains borate or boron.

In another embodiment of the present invention, the container may be a pre-filled syringe. The pre-filled syringe may be made up of a material having at least one non-glass component. The barrel of the pre-filled syringe can preferably be made up of appropriate plastic or polymeric material. In a preferred aspect, the syringe comprises a barrel made up of cyclic olefin polymer, cyclic olefin copolymer, polypropylene, polycarbonate and the like. The syringe may further comprise an elastomeric tip cap, made up of material such as chloro-butyl formulation. The syringe may comprise a plunger stopper made up of rubber material such as bromo-butyl rubber.

In some embodiments, the container may be further packaged in a secondary packaging. The secondary packaging may comprise a second container such as a pouch or overwrap or film or carton. The secondary packaging may further comprise an oxygen scavenger. In one embodiment, the secondary packaging is designed to protect the solution of diltiazem from light. In preferred embodiments, the secondary packaging pouch or film or overwrap or carton is made up of a suitable light protective material such as aluminium. Non-limiting examples of the material constituting secondary packaging or secondary containers include, aluminum, various polymers and copolymers like polyamide, ethylene vinyl alcohol copolymer, etc. Aluminum based containers are preferred and include aluminium pouches, aluminium plated films, aluminium foils, aluminum laminate films, composite aluminum films co-extruded with other polymers like polyethylene, polypropylene, EVA, EMA, EAA, etc. In one preferred embodiment, the secondary container is an overwrap pouch made up of composite polymer aluminium film having PET, Nylon-6, aluminium foil, and CPP (polypropylene/ethylene block copolymer) from outside to inside, the layers being either co-extruded and/or fixed using an adhesive with the other layer. In another preferred embodiment, the secondary container is an overwrap pouch made up of PET/NY/aluminum/oxygen absorbing layer/polyethylene. In another preferred embodiment, the second container is an overwrap pouch made up of PET/NY/aluminum/oxygen absorbing layer/polypropylene. In another preferred embodiment, the second container is an overwrap pouch made up of PET/NY/AL/OA/CPP. In some preferred embodiments, the dosage form may further comprise an oxygen scavenger, which may be placed in between the infusion container and the second overwrap container or in some embodiments, the overwrap pouch may have a layer of oxygen absorbing material which acts as an oxygen scavenger, such as fused silica bags or iron containing adsorbents like iron oxide and the like. The oxygen scavenger or oxygen scavenging layer material may be a suitable material capable of quickly absorbing oxygen and having good oxygen absorbing capacity and heat resistance. Non-limiting examples of such oxygen scavenging materials include iron, silica, charcoal, etc. Preferably the oxygen scavenging material is an iron based material. In one embodiment, the oxygen scavenger may be an iron based self-reacting type or iron based water dependent type oxygen scavenger/absorber. In one embodiment, the space between the infusion container and secondary overwrap container or pouch is filled with an inert gas such as nitrogen or argon.

In one embodiment, the present invention provides a process for the preparation of stable, ready-to-infuse parenteral dosage form, wherein said process comprises: a) taking water for injection in an amount of 80% of the batch size in a container; b) adding suitable excipients including hydroxypropyl betacyclodextrin with or without an alcoholic solvent to the container and mixing; c) adding and dissolving the diltiazem hydrochloride to the above mixture; d) checking the pH of the mixture and adjusting the pH of the solution to pH in the range of 3.5-5 by adding a suitable pH-adjusting agent, if required; e) then adjusting the volume up to 100% using water for injection; f) filtering the above solution by using 0.2 micron membrane filter, and filling the solution in a flexible infusion bag and stoppering the bag.

In a further embodiment, the present invention provides a process for preparation of a stable, ready-to-infuse parenteral dosage form, wherein the addition of a pH-adjusting agent may not be required where the pH of the solution is automatically adjusted in the range of pH 3.5-5.

In an embodiment, said infusion bag may be autoclaved at 121° C. for 15 minutes or may be kept unautoclaved. In another embodiment, said bag may be overwrapped using a suitable aluminium pouch that may optionally be filled with an oxygen scavenger and/or nitrogen gas.

In one embodiment, the present invention provides a method for the treatment of atrial fibrillation ,atrial flutter, or paroxysmal supraventricular tachycardia, by administering a stable parenteral dosage form of diltiazem, which comprises a ready-to-infuse aqueous solution of diltiazem.

In another embodiment, the present invention provides the use of a stable parenteral dosage form of diltiazem comprising a ready-to-infuse aqueous solution of diltiazem for the treatment of atrial fibrillation, atrial flutter, or paroxysmal supraventricular tachycardia.

In a further embodiment, the present invention provides a method for temporary control of rapid ventricular rate in atrial fibrillation or atrial flutter, by using a stable parenteral dosage form of diltiazem, comprising a ready-to-infuse aqueous solution of diltiazem.

In another embodiment, the present invention provides a method for administering a therapeutically effective amount of diltiazem to a patient in need thereof or a person suffering from an indication where diltiazem can be administered.

In yet another embodiment, the present invention provides a stable, ready-to-infuse, aqueous parenteral dosage form comprising diltiazem or its pharmaceutically acceptable salt, a pH-adjusting agent to provide a pH in the range of about 3.5 to about 5, at least one pharmaceutically acceptable stabilizer selected from a group comprised of hydroxypropyl derivatives of cyclodextrin, for use in treatment of atrial fibrillation, atrial flutter, or paroxysmal supraventricular tachycardia (“PSVT”).

In yet another embodiment, the present invention provides a stable, ready-to-infuse, aqueous parenteral dosage form comprising diltiazem or its pharmaceutically acceptable salt, a pH-adjusting agent to provide a pH in the range of about 3.5 to about 5, at least one pharmaceutically acceptable stabilizer selected from a group comprised of hydroxypropyl derivatives of cyclodextrin and an infusion container filled with said aqueous solution, for use in treatment of atrial fibrillation, atrial flutter, or paroxysmal supraventricular tachycardia.

In one embodiment, the present invention provides the use of a diltiazem hydrochloride ready-to-infuse dosage form according to the present invention for control of ventricular response in patients with atrial fibrillation or atrial flutter or conversion to sinus rhythm in patients with PSVT.

In the context of this specification “comprising” is to be interpreted as “including”. Aspects of the invention comprising certain elements are also intended to extend to alternative embodiments “consisting” or “consisting essentially” of the relevant elements.

Where technically appropriate, embodiments of the invention may be combined. Embodiments are described herein as comprising certain features/elements. The disclosure also extends to separate embodiments consisting or consisting essentially of said features/elements.

Any embodiments specifically and explicitly recited herein may form the basis of a disclaimer either alone or in combination with one or more further embodiments.

Hereinafter, the invention will be more specifically described by way of Examples. The examples are not intended to limit the scope of the invention and are merely used as illustrations.

EXAMPLES

### Example 1

Different exemplary batches (1A-1G) were prepared with varying concentrations of hydroxypropyl-β-cyclodextrin. The batch manufacturing was done by initiating it with 80% batch size of water for injection followed by the addition of all the excipients and addition of diltiazem hydrochloride. The compositions were adjusted to the desired pH and the volume was made up to 100%.

TABLE I
Aqueous compositions of diltiazem:

| | Batch No | | | | | | |
|---|---|---|---|---|---|---|---|
| Components/ | 1A | 1B | 1C | 1D | 1E | 1F | 1G |
| Stabilizer | Concentration (mg/ml) | | | | | | |
| Diltiazem HCl | 1 | 1 | 1 | 1 | 1 | 1 | 1 |
| Citric Acid | 0.15 | 0.15 | 0.15 | 0.15 | 0.15 | 0.15 | 0.15 |
| monohydrate | | | | | | | |
| Sodium Citrate | 0.13 | 0.13 | 0.13 | 0.13 | 0.13 | 0.13 | 0.13 |
| Sorbitol Powder | 10 | 10 | 10 | 10 | 10 | 10 | 10 |
| Dextrose Anhydrous | 40 | 40 | 40 | 40 | 40 | 40 | 40 |
| Hydroxypropyl | 0 | 0.5 | 2 | 5 | 10 | 20 | 50 |
| Betacyclodextrin | | | | | | | |
| Hydrochloric acid | q.s. to pH 3.9 | | | | | | |
| Sodium Hydroxide | q.s. to pH 3.9 | | | | | | |
| Water for Injection | q.s. to 1 ml | | | | | | |
| (WFI) | | | | | | | |

TABLE II
Stress stability data of different batches prepared in Table I:

| | | | | | | | Cis(+) | | | | Total | |
|---|---|---|---|---|---|---|---|---|---|---|---|---|
| | Hydroxypropyl | Desacetyl Diltiazem | | | Acetoxy | | hydroxy | | Maximum | | impurities* | |
| | beta | HCl (%) | | | lactum (%) | | lactum (%) | | Unknown (%) | | (%) | |
| Batch | cyclodextrin | | 7 Days/ | Change | | 7 Days/ | | 7 Days/ | | 7 Days/ | | 7 Days/ |
| No | (mg/ml) | 0 | 40° C. | (Δ/7 day) | 0 | 40° C. | 0 | 40° C. | 0 | 40° C. | 0 | 40° C. |
| 1A | 0 | 0.04 | 0.60 | 0.56 | ND | ND | ND | ND | 0.02 | 0.013 | 0.04 | 0.03 |
| 1B | 0.5 | 0.04 | 0.55 | 0.51 | ND | ND | ND | ND | 0.02 | 0.013 | 0.04 | 0.03 |
| 1C | 2 | 0.05 | 0.59 | 0.54 | ND | ND | ND | ND | 0.01 | 0.013 | 0.04 | 0.03 |
| 1D | 5 | 0.03 | 0.67 | 0.64 | ND | ND | ND | ND | 0.02 | 0.017 | 0.03 | 0.04 |
| 1E | 10 | 0.03 | 0.43 | 0.40 | ND | ND | ND | ND | 0.02 | 0.019 | 0.04 | 0.04 |
| 1F | 20 | 0.03 | 0.40 | 0.37 | ND | ND | ND | ND | 0.02 | 0.016 | 0.04 | 0.04 |
| 1G | 50 | 0.03 | 0.35 | 0.32 | ND | 0.001 | ND | ND | 0.02 | 0.017 | 0.05 | 0.05 |

Note:
*Total Impurities excluding desacetyl diltiazem HCL; ND: Not Detected.

Observation: It was observed that with the increase in concentration of hydroxypropyl-β-cyclodextrin (HP-β-CD), there was an improvement in stability of the formulation. Particularly, the concentration of 10 mg/ml or more than 10 mg/ml showed a positive effect on stability of diltiazem hydrochloride and the stability was improved as the concentration of HP-β-CD was increased.

### Example 2

Different exemplary batches (2A-2L) were prepared with varying pH of the aqueous solutions of diltiazem hydrochloride. The batch manufacturing was done by initiating it with 80% batch size of water for injection followed by addition of all the excipients and addition of diltiazem hydrochloride. The compositions were adjusted to the desired pH and the volume was made up to 100%. Half of the samples from each batch were autoclaved at 121° C. for 15 minutes, and remaining were kept unautoclaved.

TABLE III
Aqueous compositions of diltiazem:

| | Batch No | | | | | | | | | | | |
|---|---|---|---|---|---|---|---|---|---|---|---|---|
| | Concentration (mg/ml) | | | | | | | | | | | |
| Ingredients | 2A | 2B | 2C | 2D | 2E | 2F | 2G | 2H | 2I | 2J | 2K | 2L |
| Diltiazem HCl | 1 | | | | | | | | | | | |
| Ethanol | 13.5 | | | | | | | | | | | |
| Hydroxypropyl | 10 | | | | | | | | | | | |
| Betacyclodextrin | | | | | | | | | | | | |
| Hydrochloric acid | q.s. | q.s. | q.s. | q.s. | q.s. | q.s. | q.s. | q.s. | q.s. | q.s. | q.s. | q.s. |
| Sodium Hydroxide | q.s. | q.s. | q.s. | q.s. | q.s. | q.s. | q.s. | q.s. | q.s. | q.s. | q.s. | q.s. |
| Water for | q.s. | q.s. | q.s. | q.s. | q.s. | q.s. | q.s. | q.s. | q.s. | q.s. | q.s. | q.s. |
| Injection (WFI) | | | | | | | | | | | | |
| PH | 3.2 | 3.3 | 3.4 | 3.5 | 3.7 | 3.9 | 4.1 | 4.3 | 4.4 | 4.5 | 5.0 | 7.0 |

TABLE IV
Stress stability data of unautoclaved sample from different batches Table III:

| | | Desacetyl Diltiazem HCl | | | Acetoxy | | Cis(+) hydroxy | | Maximum | | Total Impurity* | |
|---|---|---|---|---|---|---|---|---|---|---|---|---|
| | | (%) | | | lactum (%) | | lactum (%) | | unknown (%) | | (%) | |
| Batch | | | 7 Day/ | Change/ | | 7 Day/ | | 7 Day/ | | 7 Day/ | | 7 Day/ |
| No. | PH | 0 | 40° C. | 7 day | 0 | 40° C. | 0 | 40° C. | 0 | 40° C. | 0 | 40° C. |
| 2A | 3.2 | 0.20 | 0.96 | 0.76 | ND | 0.05 | 0.003 | 0.005 | 0.07 | 0.07 | 0.17 | 0.20 |
| 2B | 3.3 | 0.18 | 0.88 | 0.70 | ND | ND | 0.003 | 0.005 | 0.07 | 0.08 | 0.17 | 0.20 |
| 2C | 3.4 | 0.15 | 0.73 | 0.58 | ND | ND | 0.002 | 0.009 | 0.06 | 0.06 | 0.17 | 0.18 |
| 2D | 3.5 | 0.15 | 0.67 | 0.52 | ND | 0.005 | ND | 0.003 | 0.06 | 0.06 | 0.16 | 0.17 |
| 2E | 3.7 | 0.13 | 0.58 | 0.45 | ND | ND | 0.004 | 0.003 | 0.07 | 0.06 | 0.18 | 0.16 |
| 2F | 3.9 | 0.12 | 0.56 | 0.44 | ND | ND | 0.006 | 0.006 | 0.05 | 0.06 | 0.17 | 0.18 |
| 2G | 4.1 | 0.12 | 0.56 | 0.44 | ND | ND | ND | ND | 0.06 | 0.06 | 0.16 | 0.16 |
| 2H | 4.3 | 0.14 | 0.58 | 0.44 | ND | ND | 0.004 | 0.006 | 0.06 | 0.05 | 0.16 | 0.17 |
| 2I | 4.4 | 0.14 | 0.60 | 0.46 | ND | ND | 0.003 | 0.002 | 0.06 | 0.06 | 0.16 | 0.16 |
| 2J | 4.5 | 0.15 | 0.62 | 0.47 | ND | ND | 0.003 | 0.005 | 0.06 | 0.05 | 0.17 | 0.16 |
| 2K | 5 | 0.18 | 0.70 | 0.52 | ND | ND | 0.007 | ND | 0.06 | 0.05 | 0.15 | 0.14 |
| 2L | 7 | 5.14 | 9.89 | 4.75 | ND | 0.012 | ND | 0.003 | 0.07 | 0.20 | 0.17 | 0.31 |

Note:
*Total Impurities excluding desacetyl diltiazem HCL; ND: Not Detected.

TABLE V
Stress stability data of autoclaved sample from different batches of Table III:

| | | Desacetyl Diltiazem HCl | | | Acetoxy | | Cis(+) hydroxy | | Maximum | | Total Impurity* | |
|---|---|---|---|---|---|---|---|---|---|---|---|---|
| | | (%) | | | lactum (%) | | lactum (%) | | unknown (%) | | (%) | |
| Batch | | | 7 Day/ | Change/ | | 7 Day/ | | 7 Day/ | | 7 Day/ | | 7 Day/ |
| No. | PH | 0 | 40° C. | 7 day | 0 | 40° C. | 0 | 40° C. | 0 | 40° C. | 0 | 40° C. |
| 2A | 3.2 | 0.83 | 1.68 | 0.85 | ND | ND | 0.002 | 0.005 | 0.07 | 0.05 | 0.16 | 0.17 |
| 2B | 3.3 | 0.82 | 1.52 | 0.70 | ND | 0.007 | 0.003 | 0.002 | 0.06 | 0.08 | 0.16 | 0.21 |
| 2C | 3.4 | 0.73 | 1.38 | 0.65 | 0.003 | ND | ND | 0.012 | 0.06 | 0.07 | 0.16 | 0.18 |
| 2D | 3.5 | 0.71 | 1.25 | 0.54 | ND | ND | ND | 0.004 | 0.07 | 0.06 | 0.16 | 0.17 |
| 2E | 3.7 | 0.70 | 1.20 | 0.50 | ND | ND | 0.004 | 0.008 | 0.07 | 0.06 | 0.17 | 0.19 |
| 2F | 3.9 | 0.77 | 1.26 | 0.49 | ND | ND | 0.007 | ND | 0.06 | 0.06 | 0.16 | 0.18 |
| 2G | 4.1 | 0.91 | 1.40 | 0.49 | ND | ND | 0.005 | 0.009 | 0.07 | 0.06 | 0.18 | 0.19 |
| 2H | 4.3 | 1.00 | 1.49 | 0.49 | ND | ND | 0.006 | 0.004 | 0.07 | 0.07 | 0.19 | 0.20 |
| 2I | 4.4 | 1.06 | 1.51 | 0.45 | 0.002 | ND | 0.007 | 0.009 | 0.06 | 0.06 | 0.19 | 0.19 |
| 2J | 4.5 | 1.09 | 1.58 | 0.49 | ND | ND | 0.006 | ND | 0.07 | 0.05 | 0.19 | 0.16 |
| 2K | 5 | 1.21 | 1.67 | 0.46 | ND | ND | 0.004 | 0.004 | 0.05 | 0.06 | 0.17 | 0.17 |
| 2L | 7 | 4.61 | 7.21 | 2.60 | ND | 0.004 | ND | 0.003 | 0.14 | 0.18 | 0.27 | 0.31 |

Note:
*Total Impurities excluding desacetyl diltiazem HCL; ND: Not Detected.

Observation: It was observed that the pH of the solution impacted the stability of the solution and with increase in pH, there was a gradual improvement in stability of the aqueous solution of diltiazem according to present invention. However, at a pH above 5 may be detrimental to the stability of formulation due to possible increase in formation of a known impurity.

### Example 3

Different exemplary batches (3A-3G) were prepared with varying concentrations of various excipients for the preparation of the aqueous solutions of diltiazem hydrochloride according to present invention. The batch manufacturing was done by initiating it with 80% batch size of water for injection as below:
i. a part of water for injection, 80% of the batch size was taken in a container and suitable excipients including hydroxypropyl-β-cyclodextrin with or without alcoholic solvent were added to the container and mixed;
ii. followed by adding and dissolving of diltiazem hydrochloride to the mixture of step i);
iii. checking the pH of the mixture of step ii) and adjusting the pH of the solution to pH 3.9 by adding a suitable pH-adjusting agent, if required;
iv. then the volume of the above solution of step iii) was made up to 100% using water for injection;
v. filtering the above solution of step iv) using 0.2 micron membrane filter and filled in a flexible infusion bag and stoppered.

Some of the infusion bags were autoclaved at 121° C. for 15 minutes and some were kept unautoclaved and the samples were loaded to stability study.

TABLE VI
Various aqueous compositions of diltiazem:

| | Batch No. | | | | | | |
|---|---|---|---|---|---|---|---|
| | Concentration (mg/ml) | | | | | | |
| Ingredients | 3A | 3B | 3C | 3D | 3E | 3F | 3G |
| Diltiazem Hydrochloride | 1 | 1 | 1 | 1 | 1 | 1 | 1 |
| Hydroxy propyl beta - | 10 | 35 | 35 | 10 | 10 | 35 | 35 |
| cyclodextrin | | | | | | | |
| Ethanol | — | — | — | 13.5 | 13.5 | 13.5 | 13.5 |
| Dextrose anhydrous | 40 | 40 | 40 | — | — | — | — |
| Sorbitol | 10 | 10 | 10 | — | — | — | — |
| Citric acid monohydrate | 0.15 | 0.15 | — | 0.15 | — | 0.15 | — |
| Sodium Citrate dihydrate | 0.13 | 0.13 | — | 0.13 | — | 0.13 | — |
| Hydrochloric acid | q.s. to | — | | q.s to adjust pH | | — | |
| | adjust pH | | | | | | |
| Sodium Hydroxide | q.s. to | — | | q.s to adjust pH | | — | |
| | adjust pH | | | | | | |
| pH | 3.9 | As such | 3.9 | 3.9 | 3.9 | As such | 3.9 |
| Water for Injection | q.s | q.s | q.s | q.s | q.s | q.s | q.s |

Samples from Example 3 were loaded for long term stability under refrigerated and accelerated stability. The stability details of batch 3F & G are provided in below Tables:

TABLE VII
Stability data of unautoclaved samples from Table VI:

| | | | | | Des D. | | | | |
|---|---|---|---|---|---|---|---|---|---|
| Batch | Stability | | | Des. | (Δ/ | Max. | Tot. | | Cis. HL |
| No. | Cond. | Stage | Assay | D | month) | Unk. (%) | Imp. | Ace. L | Imp |
| 3F | 2-8° C. | 0 | 99.75 | 0.05 | 0.04 | 0.03 | 0.08 | ND | ND |
| | | 6 | 99.17 | 0.33 | | 0.03 | 0.05 | ND | ND |
| | | 12 | 99.56 | 0.55 | | 0.03 | 0.06 | ND | ND |
| | | 24 | 98.67 | 1.04 | | 0.02 | 0.05 | NA | NA |
| | 25° C./ | 0 | 99.75 | 0.05 | 0.38 | 0.03 | 0.08 | ND | ND |
| | 40% | 1 | 101.07 | 0.46 | | 0.02 | 0.06 | ND | ND |
| | RH | 3 | 98.77 | 1.25 | | 0.02 | 0.02 | ND | ND |
| | | 6 | 97.81 | 2.30 | | 0.02 | 0.04 | ND | ND |
| | | 12 | 95.91 | 4.65 | | 0.02 | 0.06 | ND | ND |
| 3G | 2-8° C. | 0 | 99.46 | 0.06 | 0.04 | 0.05 | 0.16 | ND | ND |
| | | 6 | 99.41 | 0.32 | | 0.05 | 0.17 | ND | ND |
| | | 12 | 99.98 | 0.60 | | 0.05 | 0.17 | NA | NA |
| | 25° C./ | 0 | 99.46 | 0.06 | 0.39 | 0.05 | 0.16 | ND | ND |
| | 40% | 1 | 98.71 | 0.40 | | 0.05 | 0.15 | ND | ND |
| | RH | 3 | 98.32 | 1.20 | | 0.06 | 0.19 | 0.002 | ND |
| | | 6 | 97.23 | 2.40 | | 0.06 | 0.17 | 0.003 | 0.001 |
| | | 12 | 95.31 | 5.07 | | 0.04 | 0.11 | NA | NA |

Note:
Des. D: desacetyl diltiazem HCl impurity;
Δ/month: % Degradation rate (Δ/month);
Ace. L: acetoxy lactum impurity;
Tot. Imp.*: Total Impurities (excluding desacetyl diltiazem HCL impurity);
Cis. HL Imp.: Cis (+) hydroxy lactum impurity;
Max. Unk. (%): Max. Unknown Impurities (%);
NA: Not analysed

TABLE VIII
Stability data of autoclaved samples from Table VI:

| | | | | | Des D. | | | | |
|---|---|---|---|---|---|---|---|---|---|
| Batch | Stability | | | | (Δ/ | Max. | Tot. | | Cis. HL |
| No. | Cond. | Stage | Assay | Des. D | month) | Unk. (%) | Imp. | Ace. L | Imp |
| 3F | 2-8° C. | 0 | 97.75 | 1.17 | 0.04 | 0.03 | 0.08 | ND | ND |
| | | 6 | 97.67 | 1.38 | | 0.03 | 0.07 | ND | ND |
| | | 12 | 97.35 | 2.11 | | 0.02 | 0.07 | NA | NA |
| | 25° C./ | 0 | 97.75 | 1.17 | 0.37 | 0.03 | 0.08 | ND | ND |
| | 40% | 1 | 98.92 | 1.60 | | 0.03 | 0.06 | ND | ND |
| | RH | 3 | 96.44 | 2.33 | | 0.02 | 0.04 | ND | ND |
| | | 6 | 95.47 | 3.38 | | 0.03 | 0.07 | ND | ND |
| | | 12 | 94.46 | 5.64 | | 0.03 | 0.07 | ND | ND |
| 3G | 2-8° C. | 0 | 98.65 | 0.62 | 0.05 | 0.05 | 0.11 | ND | ND |
| | | 6 | 97.1 | 0.95 | | 0.05 | 0.16 | 0.003 | 0.001 |
| | | 12 | 97.59 | 1.19 | | 0.05 | 0.16 | NA | NA |
| | 25° C./ | 0 | 98.65 | 0.62 | 0.38 | 0.05 | 0.11 | ND | ND |
| | 40% | 1 | 98.07 | 1.00 | | 0.05 | 0.14 | 0.002 | 0.002 |
| | RH | 3 | 98.13 | 1.72 | | 0.06 | 0.19 | 0.004 | 0.001 |
| | | 6 | 95.54 | 2.91 | | 0.05 | 0.16 | 0.003 | 0.002 |
| | | 12 | 94.72 | 5.72 | | 0.06 | 0.16 | NA | NA |

Des. D: desacetyl diltiazem HCl impurity;
Δ/month: % Degradation rate (Δ/month);
Ace. L: acetoxy lactum impurity;
Tot. Imp.*: Total Impurities (excluding desacetyl diltiazem HCl impurity);
Cis. HL Imp.: Cis (+) hydroxy lactum impurity;
Max. Unk. (%): Max. Unknown Impurities (%);
NA: Not analyzed

Observation: It was observed that components of the formulation, particularly an alcoholic solvent like ethanol and cyclic oligosaccharide like hydroxypropyl-β-cyclodextrin, have a stabilizing effect on the overall stability of the formulation at room temperature for at least 6 months and at 2-8° C. for at least 12 months.

### Example 4

Different exemplary batches (4A-4S) were prepared with varying concentrations of various excipients for the preparation of the aqueous solutions of diltiazem hydrochloride according to present invention. The batch manufacturing was done by initiating it with 80% batch size of water for injection followed by addition of all the excipients, adjusting the pH if required, followed by addition of diltiazem hydrochloride and the volume was made up to 100%. Some of the samples were subjected to autoclaving.

TABLE IX
Various aqueous compositions of diltiazem:

| | Batch No. | | | | | | | | | | | | | | | | | | |
|---|---|---|---|---|---|---|---|---|---|---|---|---|---|---|---|---|---|---|---|
| | Concentration (mg/ml) | | | | | | | | | | | | | | | | | | |
| Ingredients | 4A | 4B | 4C | 4D | 4E | 4F | 4G | 4H | 4I | 4J | 4K | 4L | 4M | 4N | 4O | 4P | 4Q | 4R | 4S |
| Diltiazem | 1 | 1 | 1 | 1 | | 1 | | | | | 1 | | | | | | | | |
| Hydrochloride | | | | | | | | | | | | | | | | | | | |

| | | | | | | | | | | | | | | |
|---|---|---|---|---|---|---|---|---|---|---|---|---|---|---|
| HPβCD | — | 35 | 35 | 10-35 | 35 | — | — | — | — | — | 10 | — | — | — |

| Ethanol | — | — | — | — | | — | — | — | — | 13.5 | — |
|---|---|---|---|---|---|---|---|---|---|---|---|
| Dex A | 40 | 40 | — | 40 | — | — | 50 | — | — | — | 40 |

| Dex M | — | — | 35 | — | — | — | — | — | — | — | | | | | | | | |
|---|---|---|---|---|---|---|---|---|---|---|---|---|---|---|---|---|---|---|
| BHA | — | — | — | — | — | — | — | — | — | 0.02 | — | — | — | — | — | — | — | — |
| BHT | — | — | — | — | — | — | — | — | — | — | 0.06 | — | — | — | — | — | — | — |
| AMS | — | — | — | — | — | — | — | — | — | — | — | 2 | — | — | — | — | — | — |
| SM | — | — | — | — | — | — | — | — | — | — | — | — | 1 | — | — | — | — | — |
| EDTA | — | — | — | — | — | — | — | — | — | — | — | — | — | 2 | — | — | — | — |
| L-Methionine | — | — | — | — | — | — | — | — | — | — | — | — | — | — | — | 10 | — | — |
| KPM | — | — | — | — | — | — | — | — | — | — | — | — | — | — | — | — | 2 | — |
| NaH₂PO₄ | — | — | — | — | — | — | — | — | — | — | — | — | — | — | — | — | — | 2 |

| | | | | | | | | | | | |
|---|---|---|---|---|---|---|---|---|---|---|---|
| Sorbitol | 10 | 10 | 10 | 10 | — | — | — | 50 | — | — | 10 |
| NaCl | — | — | — | — | 9 | 9 | — | — | — | — | — |

| | | | | | | | | | | |
|---|---|---|---|---|---|---|---|---|---|---|
| Mannitol | — | — | — | — | — | — | — | 50 | — | — |
| CAM | 0.15 | — | — | — | — | — | — | — | — | 0.15 |
| SCD | 0.13 | — | — | — | — | — | — | — | — | 0.13 |

| | | |
|---|---|---|
| HCL | q.s. to adjust pH | — |
| NaOH | q.s. to adjust pH | — |
| pH | 3.9 | As such |
| WFI | q.s. | q.s. to 1 ml |

Note:
HP-β-CD: hydroxypropyl-β-etacyclodextrin; SCD: sodium citrate dihydrate; CAM: citric acid monohydrate; HCL: hydrochloric acid; NaOH: sodium hydroxide; WFI: water for injection; NaCl: sodium chloride; NaH₂PO₄: dodium dihydrogen phosphate; KPM: potassium phosphate monobasic; EDTA: disodium edetate; SM: dodium metabisulfite; AMS: ammonium sulfate; BHT: butylated hydroxyl tolune; BHA: butylated hydroxyl anisole; Dex M: dextrose monohydrate; Dex A: dextrose anhydrous.

Various samples from example 4 were loaded for stability analysis, data of samples 4A, 4B and 4C are provide in below table X and XI comparing the presence and absence of hydroxypropyl-β-cyclodextrin in the aqueous formulations of diltiazem hydrochloride.

TABLE X
Stability data of unautoclaved samples from Table IX:

| | | | | | Des D. | Max. | | | |
|---|---|---|---|---|---|---|---|---|---|
| Batch | Stability | | | Des. | (Δ/ | Unk. | Tot. | | Cis. HL |
| No. | Cond. | Stage | Assay | D | month) | (%) | Imp.* | Ace. L | Imp |
| 4A | 2-8° C. | 0 | 99.82 | 0.10 | 0.10 | 0.02 | 0.04 | 0.001 | ND |
| | | 1 | 100.02 | 0.18 | | 0.02 | 0.02 | ND | ND |
| | | 3 | 100.41 | 0.38 | | 0.02 | 0.07 | ND | ND |
| | | 6 | 100.74 | 0.73 | | 0.02 | 0.05 | ND | ND |
| | | 12 | 99.64 | 1.43 | | 0.03 | 0.07 | ND | ND |
| | | 24 | 99.73 | 2.57 | | 0.04 | 0.06 | NA | NA |
| | 25° C./ | 0 | 99.82 | 0.10 | 0.84 | 0.02 | 0.04 | 0.001 | ND |
| | 40% | 1 | 99.47 | 1.00 | | 0.02 | 0.03 | ND | ND |
| | RH | 3 | 98.24 | 2.75 | | 0.02 | 0.06 | ND | ND |
| | | 6 | 97.46 | 5.12 | | 0.02 | 0.06 | ND | 0.001 |
| 4B | 2-8° C. | 0 | 100.94 | 0.06 | 0.05 | 0.03 | 0.07 | ND | ND |
| | | 6 | 102.47 | 0.35 | | 0.03 | 0.08 | ND | ND |
| | | 12 | 100.69 | 0.64 | | 0.03 | 0.07 | NA | NA |
| | 25° C./ | 0 | 100.94 | 0.06 | 0.39 | 0.03 | 0.07 | ND | ND |
| | 40% | 1 | 101.46 | 0.40 | | 0.02 | 0.05 | ND | ND |
| | RH | 3 | 100.36 | 1.21 | | 0.02 | 0.07 | 0.001 | ND |
| | | 6 | 99.41 | 2.41 | | 0.03 | 0.07 | 0.002 | ND |
| | | 12 | 93.39 | 5.28 | | 0.02 | 0.08 | NA | NA |
| 4C | 2-8° C. | 0 | 99.86 | 0.05 | 0.05 | 0.02 | 0.02 | NA | NA |
| | | 3 | 100.30 | 0.19 | | 0.02 | 0.02 | NA | NA |
| | | 6 | 100.19 | 0.32 | | 0.02 | 0.03 | NA | NA |
| | | 12 | 101.40 | 0.65 | | 0.01 | 0.01 | NA | NA |
| | 25° C./ | 0 | 99.86 | 0.05 | 0.45 | 0.02 | 0.02 | NA | NA |
| | 40% | 1 | 100.10 | 0.41 | | 0.01 | 0.02 | NA | NA |
| | RH | 3 | 99.05 | 1.36 | | 0.01 | 0.03 | NA | NA |
| | | 6 | 97.76 | 2.69 | | 0.02 | 0.04 | NA | NA |
| | | 12 | 96.23 | 5.47 | | 0.01 | 0.02 | NA | NA |

Note:
Des. D: desacetyl diltiazem HCl impurity;
Δ/month: desacetyl diltiazem HCl % Degradation rate (Δ/month);
Ace. L: acetoxy lactum impurity;
Tot. Imp.*: Total Impurities (excluding desacetyl diltiazem HCl impurity);
Cis. HL Imp.: Cis (+) hydroxy lactum impurity;
Max. Unk. (%): Max. Unknown Impurities (%);
NA: Not analysed

TABLE XI
Stability data of autoclaved samples from Table IX:

| | | | | | Des D. | Max. | | | |
|---|---|---|---|---|---|---|---|---|---|
| Batch | Stability | | | Des. | (Δ/ | Unk. | Tot. | | Cis. HL |
| No. | Cond. | Stage | Assay | D | month) | (%) | Imp.* | Ace. L | Imp |
| 4A | 2-8° C. | 0 | 97.91 | 1.22 | 0.10 | 0.02 | 0.07 | 0.003 | ND |
| | | 1 | 97.37 | 1.30 | | 0.01 | 0.04 | ND | ND |
| | | 3 | 98.49 | 1.52 | | 0.02 | 0.08 | 0.002 | ND |
| | | 6 | 99.36 | 1.75 | | 0.02 | 0.08 | 0.003 | 0.002 |
| | | 12 | 98.19 | 2.41 | | 0.03 | 0.10 | ND | ND |
| | | 24 | 98.45 | 3.52 | | 0.03 | 0.09 | NA | NA |
| | 25° C./ | 0 | 97.91 | 1.22 | 0.83 | 0.02 | 0.07 | 0.003 | ND |
| | 40% | 1 | 97.31 | 2.11 | | 0.02 | 0.04 | ND | ND |
| | RH | 3 | 96.74 | 3.85 | | 0.02 | 0.08 | 0.003 | ND |
| | | 6 | 96.17 | 6.20 | | 0.02 | 0.08 | ND | ND |
| 4B | 2-8° C. | 0 | 99.91 | 0.70 | 0.05 | 0.02 | 0.06 | ND | ND |
| | | 6 | 100.19 | 0.92 | | 0.02 | 0.06 | 0.002 | ND |
| | | 12 | 100.61 | 1.25 | | 0.02 | 0.07 | NA | NA |
| | 25° C./ | 0 | 99.91 | 0.70 | 0.40 | 0.02 | 0.06 | ND | ND |
| | 40% | 1 | 100.28 | 1.13 | | 0.03 | 0.08 | ND | ND |
| | RH | 3 | 99.59 | 1.86 | | 0.03 | 0.08 | 0.001 | ND |
| | | 6 | 98.16 | 3.14 | | 0.03 | 0.08 | 0.002 | ND |
| | | 12 | 94.81 | 5.86 | | 0.02 | 0.10 | NA | NA |
| 4C | 2-8° C. | 0 | 99.07 | 0.79 | 0.05 | 0.01 | 0.03 | NA | NA |
| | | 3 | 98.73 | 0.93 | | 0.02 | 0.04 | NA | NA |
| | | 6 | 98.10 | 1.07 | | 0.01 | 0.05 | NA | NA |
| | | 12 | 99.50 | 1.42 | | 0.02 | 0.02 | NA | NA |
| | 25° C./ | 0 | 99.07 | 0.79 | 0.45 | 0.01 | 0.03 | NA | NA |
| | 40% | 1 | 99.32 | 1.16 | | 0.01 | 0.01 | NA | NA |
| | RH | 3 | 97.43 | 2.11 | | 0.01 | 0.04 | NA | NA |
| | | 6 | 96.40 | 3.42 | | 0.01 | 0.06 | NA | NA |
| | | 12 | 95.51 | 6.21 | | 0.02 | 0.03 | NA | NA |

Note:
Des. D: desacetyl diltiazem HCl impurity;
Δ/month: desacetyl diltiazem HCl % Degradation rate (Δ/month);
Ace. L: acetoxy lactum impurity;
Tot. Imp.*: Total Impurities (excluding desacetyl diltiazem HCl impurity);
Cis. HL Imp.: Cis (+) hydroxy lactum impurity;
Max. Unk. (%): Max. Unknown Impurities (%);
NA: Not analysed

Observation: It was observed that the components of the formulations according to present invention were stable for a prolonged period of time with cyclodextrin and various other solvents and osmogen. Cyclodextrin containing formulations of diltiazem were relatively found to be more stable.

1. A parenteral dosage form comprising a ready-to-infuse, stable aqueous solution comprising diltiazem or a pharmaceutically acceptable salt thereof and a cyclic oligosaccharide as a stabilizer.

2. The parenteral dosage form according to claim 1, wherein the cyclic oligosaccharide comprises a macrocyclic ring of glucose subunits joined by α-1,4 glycosidic bonds.

3. The parenteral dosage form according to claim 1, wherein the said dosage form is filled in an infusion container.

4. The parenteral dosage form according to claim 3, wherein said infusion container is an infusion bag, a perfusion bag, a flexible pouch, a soft bag, an infusion bottle or a pre-filled syringe.

5. The parenteral dosage form according to claim 1, wherein diltiazem or pharmaceutically acceptable salt thereof is present in a concentration range of about 0.05 mg/ml to about 2.0 mg/ml.

6. The parenteral dosage form according to claim 1, wherein said aqueous solution further comprises an alcoholic solvent at a concentration ranging from about 1.0% w/v to about 50.0% w/v.

7. The parenteral dosage form of claim 3, wherein the aqueous solution is present in a volume ranging from about 50 ml to about 500 ml in the infusion container.

8. The parenteral dosage form of claim 1, wherein said cyclic oligosaccharide stabilizer is a cyclodextrin.

9. The parenteral dosage form of claim 8, wherein the cyclodextrin stabilizer is selected from hydroxypropyl-β-cyclodextrin (HP-β-CD), methylated-β-cyclodextrin (RM-β-CD) or sulfobutylether-β-cyclodextrin (SBE-β-CD).

10. The parenteral dosage form of claim 9, wherein the cyclodextrin stabilizer is present in a concentration ranging from about 0.1% to about 50% w/v.

11. (canceled)

12. The parenteral dosage form of claim 1, wherein said aqueous solution further comprises a pH-adjusting agent to provide a pH in the range of about 3.5 to about 5.

13. A parenteral dosage form comprising a ready-to-infuse, stable aqueous solution comprising diltiazem or a pharmaceutically acceptable salt thereof, a cyclic oligosaccharide as a stabilizer, an alcoholic solvent, and optionally a pH-adjusting agent.

14. The parenteral dosage form of claim 13, wherein the cyclic oligosaccharide stabilizer is selected from hydroxypropyl-β-cyclodextrin (HP-β-CD), methylated-β-cyclodextrin (RM-β-CD) or sulfobutylether-β-cyclodextrin (SBE-β-CD).

15. The parenteral dosage form of claim 13, wherein the alcoholic solvent is ethanol or a mixture of ethanol with another co-solvent.

16. The parenteral dosage form according to claim 13, wherein the diltiazem or pharmaceutical acceptable salt thereof is in a concentration range of about 0.05 to about 2.0 mg/ml, the cyclic oligosaccharide is hydroxypropyl-β-cyclodextrin in a concentration range of about 0.1 to about 50% w/v, and the solution has a pH ranging from about 3.7 to about 4.5.

17. The parenteral dosage form according to claim β, wherein the diltiazem or pharmaceutical acceptable salt thereof is in a concentration range of about 0.1 to 2.0 mg/ml, the cyclic oligosaccharide is hydroxypropyl-β-cyclodextrin in a concentration range of about 0.1 to 10% w/v, and the solution has a pH of about 3.9.

18. The parenteral dosage form according to claim 13, wherein the diltiazem or pharmaceutical acceptable salt thereof is in a concentration range of about 0.1 to 2.0 mg/ml, the cyclic oligosaccharide is hydroxypropyl-β-cyclodextrin in a concentration range of about 0.1 to 10% w/v, the alcoholic solvent is ethanol in a concentration range of about 1 to about 10% w/w and the solution has a pH of about 3.9.

19. The parenteral dosage form of claim 13, wherein the aqueous solution has less than 10% of diltiazem desacetyl or salt thereof when said aqueous solution is stored at 2-8° C. for at least 12 months.

20. The parenteral dosage form of claim 13, wherein the aqueous solution has less than 10% of diltiazem desacetyl or salt thereof when said aqueous solution is stored at 25° C./40% RH for at least 6 months.

21. The parenteral dosage form of claim 13, wherein said dosage form is terminally sterilized by autoclaving.

22. A method for treating atrial fibrillation, atrial flutter, or paroxysmal supraventricular tachycardia, the method comprising administering a stable parenteral dosage form according to claim 13 to a patient in need thereof.

23. (canceled)

24. A parenteral dosage form comprising a ready-to-infuse, stable aqueous solution comprising diltiazem or its pharmaceutically acceptable salt and a cyclic oligosaccharide as a stabilizer, wherein in the dosage form the level of desacetyl diltiazem or salt thereof is not more than about 10% w/w of diltiazem or a pharmaceutically acceptable salt thereof when stored for at least 12 months at 2-8° C. or when stored for at least 6 months at 25° C./40% RH.

25-27. (canceled)

28. The parenteral dosage according to claim 13, wherein said dosage form is prepared by a process comprising of:
a) taking water for injection in an amount of 80% of a batch size in a container;
b) adding excipients including the cyclic oligosaccharide to the container and mixing;
c) adding and dissolving diltiazem hydrochloride to the mixture;
d) adjusting the pH of the solution to a pH in the range of about 3.5 to about 5 by adding the pH-adjusting agent, if required;
e) making up the volume to 100% using water for injection;
f) filtering the solution of step e) using a 0.2 micron membrane filter.

29-31. (canceled)

32. A method for controlling the level of desacetyl diltiazem or its salt impurity in a ready-to-infuse parenteral dosage form of diltiazem or its pharmaceutically acceptable salt by using a cyclic oligosaccharide stabilizer, wherein in the dosage form the level of desacetyl diltiazem or salt thereof is not more than 6% w/w of diltiazem or a pharmaceutically acceptable salt thereof when stored for at least 12 months at 2-8° C. or when stored for at least 6 months at 25° C./40% RH.

33 -55. (canceled)
